(19) United States
(12) Patent Application Publication
(10) Pub. No.: US 2022/0071952 A1
(43) Pub. Date: Mar. 10, 2022

(54) METHODS OF TREATING AND PREVENTING HEPATOTOXICITY

(71) Applicant: Natreon, Inc., New Brunswick, NJ (US)

(72) Inventor: Sanyasi R Kalidindi, Monroe, NJ (US)

(73) Assignee: Natreon, Inc., New Brunswick, NJ (US)

(21) Appl. No.: 17/466,815

(22) Filed: Sep. 3, 2021

Related U.S. Application Data
(60) Provisional application No. 63/074,533, filed on Sep. 4, 2020.

(51) Int. Cl. A61K 31/366; A61P 1/16

(52) U.S. Cl. CPC A61K 31/366; A61P 1/16

(57) ABSTRACT

This invention is directed to methods of treating and/or preventing hepatotoxicity and providing hepatoprotection in a subject with dibenzo-alpha-pyrones (DBPs), including 3-OH-DBP (Urolithin B), 3,8-(OH)₂-DBP (Urolithin A), and synergistic combinations of the two DBPs.

### CROSS-REFERENCE TO RELATED APPLICATIONS

This application claims the benefit of U.S. Provisional Application No. 63/074,533, filed Sep. 4, 2020. Said provisional application is incorporated by reference in its entirety herein.

### FIELD OF INVENTION

This invention relates to methods of treating and/or preventing hepatotoxicity and providing hepatoprotection with 3-hydroxy-dibenzo-alpha-pyrone (Urolithin B) and 3,8-dihydroxy-dibenzo-alpha-pyrone (Urolithin A).

### BACKGROUND

Paracetamol, also known as acetaminophen, is a non-prescription drug used widely throughout the world to treat symptoms of pain, fever, headache, osteoarthritis, and various other health problems. While generally well-tolerated, paracetamol may induce hepatotoxicity (liver damage), for instance due to overuse or an overdose.

Alcohol-related disorders present on-going, challenging health problems, with far-reaching medical, social, and economic consequences. Long-term alcohol use potentially results in serious illnesses, including alcoholic fatty liver hypertriglyceridemia, cirrhosis, cardiovascular disease, and inflammation of the pancreas. Although much progress has been made in understanding the pathogenesis of alcoholic liver disease, therapy to prevent and treat the disease is limited.

Studies on liver disease and treatments may include using a substance to induce liver damage in laboratory animals and then analyzing evidence of hepatotoxicity. Hepatotoxicity may be induced, for instance, by paracetamol or ethanol, or by other known hepatotoxic substances such as carbon tetrachloride (CCl₄). An increase in liver enzymes in the bloodstream such as alanine aminotransferase (ALT, also known as GPT, SGPT or ALAT), aspartate aminotransferase (AST, also known as GOT, SGOT, or ASAT)), ALP (alkaline phosphatase), and/or ACP (acid phosphatase) is an indicator of hepatotoxicity. Other indicators of hepatotoxicity include an increase in serum triglycerides, an increase in serum bilirubin, a decrease in total liver protein, and/or an increase in total liver weight. Also, liver damage may be evidenced by an increase in serum MDA (Malondialdehyde), which indicates increased lipid peroxidation and increased oxidative stress in the liver. Ursocol® (ursodeoxycholic acid) may help reduce hepatotoxicity.

Shilajit is a natural substance found in mountain rocks during peak summer months. It is found at high altitudes ranging from 1000 to 5000 meters. Active constituents of Shilajit include dibenzo-alpha-pyrones (DBPs, also known as “urolithins”), specifically, 3-hydroxy-dibenzo-alpha-pyrone (3-OH-DBP, Urolithin B) and 3,8-dihydroxy-dibenzo-alpha-pyrone (3,8-(OH)₂-DBP, Urolithin A) (See Trivedi et al., 2004)). Shilajit also includes for instance DBP-related metabolites, small peptides (constituting non-protein amino acids), some lipids and carrier molecules, including for instance fulvic acids, humic acids, minerals. PrimaVie® is a standardized Shilajit extract prepared by extracting shilajit with aqueous solution as is known in the art, and formulated as a potent and very safe dietary supplement, restoring the energetic balance and potentially able to prevent several diseases.

### SUMMARY OF INVENTION

The present invention is directed to methods of protecting a subject from liver damage, and methods for treating and/or preventing hepatotoxicity in a subject, with 3-OH-DBP and/or 3,8-(OH)₂-DBP, or a Shilajit composition enriched with 3-OH-DBP and/or 3,8-(OH)₂-DBP.

In an embodiment, a method of treating and/or preventing hepatotoxicity in a subject according to the present invention comprises the steps of:
a) providing a composition comprising 3-OH-dibenzo-alpha-pyrone (3-OH-DBP) and/or 3,8-(OH)₂-dibenzo-alpha-pyrone (3,8-(OH)₂-DBP); and
b) administering the composition to the subject in an effective amount to deliver the 3-OH-DBP and/or 3,8-(OH)₂-DBP to the subject's liver to act on the liver and improve one or more abnormal hepatic parameters and/or maintain one or more hepatic parameters in the subject.

In an embodiment, the effective amount comprises 3-OH-DBP, 3,8-(OH)₂-DBP, or a combined (3-OH-DBP+3,8-(OH)₂-DBP) in a daily dose of about 0.5 mg/kg of the subject's body weight to about 75 mg/kg of the subject's body weight, for instance, about 1.0-50 or about 1.5-25 mg/kg.

In an embodiment, a method of providing hepatoprotection in a subject according to the present invention comprises the steps of:
a) providing a composition comprising 3-OH-DBP and/or 3,8-(OH)₂-DBP; and
b) administering the composition to the subject in an effective amount to deliver the 3-OH-DBP and/or 3,8-(OH)₂-DBP to the tissues and bloodstream of the subject's liver to protect the liver from damage.
   In an embodiment, the effective amount comprises 3-OH-DBP, 3,8-(OH)₂-DBP, or a combined (3-OH-DBP+3,8-(OH)₂-DBP) in a daily dose of about 0.5 mg/kg of the subject's body weight to about 75 mg/kg of the subject's body weight, for instance, about 1.0-50 or about 1.5-25 mg/kg. Additional embodiments are described throughout the present specification and claims.

### DETAILED DESCRIPTION

The present invention is directed to methods for treating and/or preventing hepatotoxicity in a subject with 3-hydroxy-dibenzo-alpha-pyrone and/or 3,8-dihydroxy-dibenzo-alpha-pyrone and for providing hepatoprotection with these substances from hepatotoxicity from for instance drugs and other substances such as ethanol, paracetamol, and/or carbon tetrachloride.

The below definitions and discussion are intended to guide understanding but are not intended to be limiting with regard to other disclosures in this application. References to percentage (%) in compositions of the present invention are to the % by weight of a given component to the total weight of the composition being discussed, also signified by “w/w”, unless stated otherwise.

A “composition” of the present invention comprises 3-OH-DBP and/or 3,8-(OH)₂-DBP. In an embodiment, a composition of the present invention comprises, consists essentially of, or consists of 3-OH-DBP and/or 3,8-(OH)₂-DBP. In an embodiment, the 3-OH-DBP and/or 3,8-(OH)₂-DBP are in purified form, for instance, greater than 90% pure, greater than 95% pure, or for instance 99% pure or greater. In an embodiment, the 3-OH-DBP and/or 3,8-(OH)₂-DBP are synthesized; in an embodiment, a DBP of this invention is from a natural source. In an embodiment, a “combination” of this invention refers to 3-OH-DBP and 3,8-(OH)₂-DBP, together in a composition (for instance mixed together by simple mixing methods, with or without other substances present). In an embodiment, a combination of this invention may include 3-OH-DBP and 3,8-(OH)₂-DBP in separate compositions co-administered or to be co-administered so that the 3-OH-DBP and 3,8-(OH)₂-DBP are or will be together in the subject's body (“combined” in the body) in an effective amount to treat and/or prevent hepatotoxicity or provide hepatoprotection according to the present invention. In an embodiment, a combination according to the present invention is a “synergistic combination” providing a synergistic effect, in an embodiment greater or otherwise better than the effect achieved by 3-OH-DBP and 3,8-(OH)₂-DBP alone. While references to synergy and synergistic effects may be made throughout this application, all instances of such may not be expressly pointed out. In an embodiment, a composition of the present invention is a synergistic composition comprising a synergistic combination of 3-OH-DBP and/or 3,8-(OH)₂-DBP. In an embodiment, a synergistic combination according to this invention is a combination of 3-OH-DBP: 3,8-(OH)₂-DBP in a ratio of 1:3 to 1:7. In an embodiment, the synergistic combination is a ratio of 3-OH-DBP: 3,8-(OH)₂-DBP of 1:5, for instance as discussed in Example IV below. The term “combination” or the like is not intended to be limiting in the context of this invention. In an embodiment, a composition of this invention is PrimaVie® Shilajit (Natreon, Inc., New Brunswick, N.J.), enriched with either DBP or a combination of both DBPs. In an embodiment, said enriched Shilajit includes an amount of 3-OH-DBP and/or 3,8-(OH)₂-DBP that is increased over the amount of 3-OH-DBP and/or 3,8-(OH)₂-DBP occurring in Shilajit such as PrimaVie® Shilajit (an extract of shilajit) alone, up to amounts indicated in compositions and as effective amounts throughout this application.

A composition of the present invention may be formulated into nutraceutical or pharmaceutical dosage forms comprising for instance tablets, capsules, powders, liquids, chews, gummies, transdermals, injectables, dietary supplements, topical creams, lozenges, pills, and so forth. A composition of the present invention may further comprise one or more excipients, additives, and/or other substances, including for instance microcrystalline cellulose, croscarmellose sodium, magnesium stearate, and/or silicon dioxide; and/or a suitable aqueous solution such as a buffer solution or carboxymethyl cellulose as in the Examples below.

A “dietary supplement” according to the present invention refers to a composition of the present invention, comprising 3-OH-DBP and/or 3,8-(OH)₂-DBP as discussed throughout this application, including Shilajit enriched with DBPs, which is orally administered as an addition to a subject's diet, which when administered delivers 3-OH-DBP and/or 3,8-(OH)₂-DBP to the subject's liver (e.g. hepatic tissues, cells, circulation) and treats and/or prevents hepatotoxicity in the subject's liver, or provides hepatoprotection to the liver. In an embodiment, the dietary supplement or other composition of this invention is administered daily. In an embodiment, the dietary supplement or other composition is administered daily for at least 1 day, 1 day to 1 week, 7 days, or 1 week to 4 weeks or to 8 weeks or to 12 weeks, or chronically for at least 3 months, 6 months, 9 months, or 1 year or more, or for another period of time according to the present invention. A dietary supplement may be formulated into various forms including a powder, as discussed throughout this application. In an embodiment, PrimaVie® Shilajit, enriched with DBPs to effective amounts according to this invention, is a dietary supplement of this invention.

“Administering”, “administration”, and the like, according to the present invention refer to providing a composition of the present invention to a subject so that the 3-OH-DBP and/or 3,8-(OH)₂-DBP may reach the subject's liver including hepatic blood vessels and/or hepatic tissues and/or cells, in an amount effective for the 3-OH-DBP and/or 3,8-(OH)₂-DBP to act on the liver tissues and cells to treat and/or prevent hepatotoxicity, and/or to provide hepatoprotection.

In the present invention, an “effective amount” of 3-OH-DBP and/or 3,8-(OH)₂-DBP, “amount effective”, and the like, refers to an amount of 3-OH-DBP and/or 3,8-(OH)₂-DBP needed to reach a subject's liver including hepatic circulation and/or tissues and/or cells to treat and/or prevent hepatotoxicity and/or to provide hepatoprotection.

In an embodiment, an effective amount of 3-OH-DBP is a daily dosage of about 0.5 mg/kg body weight (of the subject) to about 100 mg/kg body weight. In an embodiment, an effective amount of 3-OH-DBP in a subject is a daily dosage of about 0.5 mg/kg body weight to about 75 mg/kg body weight, including for instance about 1 mg/kg to about 50 mg/kg, about 1.5 mg/kg to about 25 mg/kg, about 10-20 mg/kg, about 2-10 mg/kg, about 1-3 mg/kg, and other ranges. In an embodiment, an effective amount of 3-OH-DBP is a daily dosage of about 0.5, 1.0, 1.5, 2, 5, 10, 25, 50, or 75 mg 3-OH-DBP/kg of the subject's body weight. In an embodiment, an effective amount of 3,8-(OH)₂-DBP is a daily dosage of about 0.5 mg/kg body weight (of the subject) to about 100 mg/kg body weight. In an embodiment, an effective amount of 3,8-(OH)₂-DBP in a subject is a daily dosage of about 0.5 mg/kg body weight to about 75 mg/kg body weight, including for instance about 1 mg/kg to about 50 mg/kg, about 1.5 mg/kg to about 25 mg/kg, about 10-20 mg/kg, about 30-50 mg/kg, about 2-10 mg/kg, and other ranges. In an embodiment, an effective amount of 3,8-(OH)₂-DBP is a daily dosage of about 0.5, 1.0, 1.5, 2, 5, 10, 25, 50, or 75 mg 3-OH-DBP/kg of the subject's body weight. Combinations of 3-OH-DBP and 3,8-(OH)₂-DBP in any amounts for instance as described above and otherwise throughout this application may be included in this invention. In an embodiment, an effective amount is a daily dosage of a combination of about 1-20, or about 2-10, mg 3-OH-DBP/kg body weight of the subject and about 5-75, or about 10-50, mg 3,8-(OH)₂-DBP/kg body weight of the subject. In an embodiment, a daily dose for an adult human subject of this invention is a solid oral dose unit composition with about 1-5000 mg of 3-OH-DBP, 3,8-(OH)₂-DBP, or 3-OH-DBP and 3,8-(OH)₂-DBP combined (3-OH-DBP+3,8-(OH)₂-DBP), with 1-4 dose units taken per day. In an embodiment, said daily dose is for instance 10-1000 mg, 50-750 mg, 100-500 mg and any individual dosages therewithin including for instance 1, 5, 10, 50, 100, 125, 200, 250, 500 mg of 3-OH-DBP, 3,8-(OH)₂-DBP, or combined 3-OH-DBP+3,8-(OH)₂-DBP, or more. Administration of a composition according to this invention may be by the subject or by another. Administration may be oral, for instance in the form of a dietary supplement, and/or in a solid dosage form, such as a capsule, and/or through other physiologically acceptable routes such as parenteral, intramuscular, transdermal, intraperitoneal, intravenous, and so forth.

A “subject” of the present invention refers to an animal having a liver. In an embodiment, a subject is a mammal, such as a mouse, rat, dog, cat, or horse. In a preferred embodiment, a subject is a human. In an embodiment, the subject such as a human subject is healthy with a healthy liver as shown by healthy normal hepatic parameters and no abnormal hepatic parameters indicating existing hepatoxicity. In an embodiment, the subject such as a human subject has one or more abnormal hepatic parameters. In an embodiment, the subject has 1, 2, 3, 4, or more abnormal hepatic parameters indicating hepatotoxicity.

A “hepatic parameter” according to this invention refers to a measured biochemical or physical parameter (characteristic of the subject's liver) that may be used to indicate the healthy status of the liver, or the presence of hepatotoxicity, such as from exposure to a hepatotoxic substance (e.g. alcohol such as ethanol, paracetamol/acetaminophen, carbon tetrachloride) or other insult. A hepatic parameter may indicate a healthy or abnormal status for instance based on a subject's prior history of such measurements and/or based on ranges typically considered to be normal or not normal (abnormal) for the subject's species and optionally age, gender, and the like. In an embodiment, a hepatic parameter of this invention is a measure of serum marker enzymes such as ALT, AST, ALP, and/or ACP, and/or serum triglycerides, bilirubin, and/or MDA. In an embodiment, a hepatic parameter is a physical hepatic parameter, such as an assessment of relative organ (liver) weight. In an embodiment, an abnormal hepatic parameter is a hepatic parameter that is not a normal healthy hepatic parameter, for instance different from a healthy measurement from a subject's medical history, or outside of the range of healthy normal measurements for the subject's species and as desired subcategories such as gender, age, or special status.

“Treatment”, “treat”, “treating” and the like according to the present invention refer to administering an effective amount of 3-OH-DBP and/or 3,8-(OH)₂-DBP to a subject that has hepatotoxicity for instance as evidenced by an abnormal hepatic parameter. The 3-OH-DBP and/or 3,8-(OH)₂-DBP reach the subject's liver and act upon the liver's cells and tissues to improve (e.g. attenuate, reverse, and/or eliminate) the subject's hepatotoxicity (for instance as shown in the Examples below) and thereby improve abnormal hepatic parameters for instance to normal or approaching normal. In an embodiment, treatment of hepatotoxicity according to this invention refers to improving one or more hepatic parameters such as abnormal hepatic parameters during and/or after hepatotoxic insult, such as from exposure to damaging substances or events including for instance exposure of the liver to drugs or other chemicals such as paracetamol, CCl₄, or ethanol. In an embodiment, improvement of a hepatic parameter such as an abnormal hepatic parameter according to this invention refers to for instance restoring serum marker enzymes such as ALT, AST, ALP, and/or ACP, and/or serum triglycerides, bilirubin, MDA, to normal healthy levels or to levels approaching normal healthy levels for the subject or on average for the subject's species and optionally e.g. gender. Improving hepatic parameters such as abnormal hepatic parameters according to this invention may also refer to restoring total liver protein and organ size to normal healthy levels or to levels approaching normal healthy levels for instance for the individual or on average for the subject's species and optionally for instance gender. For instance, Example I Table 3 shows normal healthy levels of serum AST in Group I (51.40 IU/L), abnormal levels of serum AST in Group II (108.5 IU/L), indicating hepatotoxicity in Group II, and then restoration of AST levels to Group I levels for instance with the administration of 3,8-(OH)₂-DBP in Group IV (51.76 IU/L). Also for instance, Example II Table 5 shows normal healthy levels of MDA in Group I (5.58 nmol/ml), elevated hepatotoxic levels of MDA in Group II (9.39 nmol/ml), and then the restoration of MDA levels to levels approaching normal healthy levels after administration of 3-OH-DBP (Group III: 7.04 nmol/ml) and 3,8-(OH)₂-DBP (Group IV: 6.59 nmol/ml). In an embodiment, “treatment” and the like refer to administering 3-OH-DBP and/or 3,8-(OH)₂-DBP to subjects with on-going and/or chronic insult, for instance to subjects that routinely or frequently use or overuse alcohol or paracetamol or other substance(s) that may cause or contribute to hepatotoxicity. In an embodiment, “treatment” and the like refers to administering 3-OH-DBP and/or 3,8-(OH)₂-DBP to subjects that occasionally or sporadically use alcohol or paracetamol or other substance(s) that may cause or contribute to hepatotoxicity. In an embodiment, treatment according to this invention includes treatment of an alcohol-related disorder, and/or of drug-induced liver injury.

“Prevention”, “prevent”, “preventing” and the like according to this invention refer to administering one or both DBPs of this invention in an effective amount to a subject prior to or during hepatotoxic insult such as exposure of the liver to drugs or other chemicals such as paracetamol, CCl₄, or ethanol. In an embodiment, the subject has a healthy liver prior to hepatotoxic insult (i.e. the subject's hepatic parameters fall within normal ranges for the subject or for the subject's species and optionally e.g. gender), so that the DBP(s) reach the subject's liver and act upon the cells and tissues of the liver so that, upon receiving a hepatotoxic insult, hepatic parameters remain within normal, healthy levels, or nearer to normal healthy levels than with the insult alone, thus avoiding or minimizing hepatotoxicity from a future, potential hepatotoxic insult. In an embodiment, a subject may be administered a composition of the present invention daily. In an embodiment, a subject may be administered a composition of the present invention at least 1-7 days prior to hepatotoxic insult. In an embodiment, prevention according to this invention includes administering one or both DBPs of this invention in an effective amount to a subject during or after a hepatotoxic insult, so that the DBP(s) reach the subject's liver and help prevent damage from the insult. In an embodiment, the present invention is directed to a method of preventing hepatotoxicity by providing and administering a composition having one or both DBPs of this invention in an effective amount to maintain one or more normal hepatic parameters in the subject, for instance before, during, or after exposure to ethanol, paracetamol, carbon tetrachloride, or other hepatic insult. In an embodiment, treatment relates to existing hepatotoxicity, for instance including on-going hepatotoxicity with a hepatotoxic chemical present as a hepatotoxic insult, and optionally actively damaging the liver; or including hepatotoxicity remaining after removal of the insult. In an embodiment, prevention relates to maintaining healthy hepatic parameters and avoiding hepatotoxicity. In an embodiment, prevention according to this invention includes prevention of damage from an alcohol-related disorder, and/or from drug-induced liver injury.

“Hepatotoxicity”, “hepatotoxic”, and the like according to the present invention refer to damage to the liver, for instance in structure and/or in function. In an embodiment, hepatotoxicity is caused or caused in part by exposure of the liver to an insult such as a chemical insult. In an embodiment, hepatotoxicity is caused or caused in part by exposure of the liver to paracetamol, carbon tetrachloride (CCl₄), or ethanol. In an embodiment, hepatotoxicity is evidenced by abnormal hepatic parameters (where abnormal refers to levels outside of a normal healthy range for the individual subject or the subject's species and optionally e.g. gender). In an embodiment, normal healthy hepatic parameters according to this invention refer to for instance normal healthy levels of serum marker enzymes such as ALT, AST, ALP, and/or ACP, as well as other serum measures such as levels of triglycerides, bilirubin, and/or MDA. Also, hepatic parameters according to this invention may refer to normal healthy levels of total protein and organ size, and other parameters for instance as measured in the below Examples. In an embodiment, normal healthy levels of serum ALT, AST, ALP, triglycerides, and bilirubin may be seen for instance in Example I Table 2 Group I, below, and abnormal levels of ALT, AST, ALP, triglycerides, and bilirubin indicating hepatotoxicity seen in Table 2 Group II.

“Hepatoprotective”, “hepatoprotection” and the like according to the present invention refers to 3-OH-DBP and/or 3,8-(OH)₂-DBP acting on liver tissues and cells so that hepatotoxicity (for instance as evidenced by changes in hepatic parameters from normal to abnormal, as discussed above) is avoided or reduced. Hepatoprotection is provided by 3-OH-DBP and/or 3,8-(OH)₂-DBP for instance as shown and/or discussed in each Example below. In an embodiment, hepatoprotection is evidenced by maintaining or minimizing increases in serum ALT, AST, ALP, ACP, bilirubin, triglycerides, MDA, and/or other hepatic parameters that would otherwise increase upon hepatic insult, for instance with ethanol, CCl₄, or paracetamol.

The present invention may be further understood in connection with the following Examples and embodiments. The Examples and embodiments described throughout this application are provided to illustrate the invention and are not intended as limiting.

### Example I

The below example shows hepatoprotective activity of 3-OH-DBP and 3,8-(OH)₂-DBP and treatment and/or prevention of paracetamol-induced hepatotoxicity in mice.

### Materials and Methods

3-OH-DBP (Natreon, Inc., New Brunswick, N.J., synthesized to 99% purity w/w) was in the form of a white free flowing powder. 3,8-(OH)₂-DBP (Natreon, Inc., New Brunswick, N.J., synthesized to 99% purity w/w) was in the form of a pale green free flowing powder. Paracetamol (acetaminophen) was in the form of a white solid tablet.

### Experimental Animals

Swiss albino mice of both sexes, weighing approximately 25-30 g, were obtained from National Research Institute of Ayurveda for Drug Development (Govt. of India), Kolkata, and were housed in polypropylene cages at 22±3° C., relative air humidity of 45 to 55%, with 12.00 hr light and dark cycle (lighting on from 6:00 AM to 6:00 PM). Mice were provided standard pellet chow (carbohydrate 65.5%, protein 17.6%, fat 6.6%) and distilled water ad libitum. The mice were acclimatized for one week in the above laboratory conditions, before being used in this experiment. All experiments were conducted between 10.00 hr and 14.00 hr. Principles of laboratory animal care (NIH publication no. 85-23, revised 1985) were followed.

### Drug Protocol

The animals were randomly assigned into four groups of six animals each, as set out in Table 1. The test compounds were suspended in 0.3% CMC (Carboxymethyl Cellulose) solutions of distilled water and were administered orally at a dose of 10 mg/kg for 7 days by using an intubation canula and volume of dose was 0.1 ml/10 g body weight. Paracetamol was suspended in 0.3% CMC solutions of distilled water and was administered orally at a dose of 200 mg/kg for 7 days in mice except vehicle control group. Vehicle control group animals received equivalent volume of the vehicle (0.3% CMC) only. Table I shows the description of the groups. After 1 hr of last treatment, blood samples were collected and serum was separated.

TABLE 1
Description of groups

| Group | Administered |
|---|---|
| Group I | Vehicle control (0.3% CMC p.o. (“by mouth”)) |
| Group II | Paracetamol (200 mg/kg b.w. (“body weight”) p.o.) |
| Group III | 3-OH-DBP (10 mg/kg b.w. p.o.) + |
| | Paracetamol (200 mg/kg b.w. p.o.) |
| Group IV | 3,8-(OH)₂-DBP (10 mg/kg b.w, p.o.) + |
| | Paracetamol (200 mg/kg b.w, p.o.) |

### Biochemical Analysis

The serum samples were used for the assay of biochemical marker enzymes (ALT, AST and ALP), total protein, triglycerides, and bilirubin levels.

### Results and Discussion

Paracetamol administration [in 0.3% CMC at the dose of 200 mg/kg body weight (b.w.) orally once daily for 7 days] produced hepatotoxicity in mice as indicated by the increased levels of hepatic marker enzymes (ALT, AST), triglycerides, bilirubin and decreased levels of total protein in Group II (Table 2), as compared with Group I; and as similarly evidenced by increased serum ALT, AST, ALP, Triglycerides (p<0.001) and bilirubin (p<0.05) in Table 3.

Treatment with 3-OH-DBP (Group III, 10 mg/kg body weight) and with 3,8-(OH)₂-DBP (Group IV, 10 mg/kg body weight) significantly attenuated and reversed the paracetamol-induced hepatotoxicity as evidenced by the significant (p<0.01) decrease (Table 2) in ALT and AST levels in comparison with the paracetamol-treated group (Group II). Treatment with 3-OH-DBP (Group III, 10 mg/kg body weight) and 3,8-(OH)₂-DBP (Group IV, 10 mg/kg body weight) significantly attenuated and reversed the paracetamol-induced hepatotoxicity as evidenced by the significant (p<0.001) decrease in ALT, AST, and triglyceride levels as shown in Table 3 in comparison with the paracetamol-treated group (Group II), as well as the significant decrease in serum bilirubin (p<0.05), and of ALP by 3-OH-DBP (p<0.05). Other evidence as set out in Tables 2 and 3 provides further evidence of hepatoprotection by 3-OH-DBP and/or 3,8-(OH)₂-DBP.

TABLE 2
Biochemical Parameters After 7 Days of Treatment (Male Mice)

| | Parameters | | | | | |
|---|---|---|---|---|---|---|
| | | | | Total | | Total |
| | ALT | AST | ALP | Protein | Triglyceride | Bilirubin |
| Group | (IU/L) | (IU/L) | (IU/L) | (mg/dl) | (mg/dl) | (mg/dl) |
| Group I | 41.90 ± 13.16 | 44.95 ± 8.97 | 86.38 ± 8.50 | 6.47 ± 0.71 | 71.59 ± 0.81 | 0.22 ± 0.07 |
| Vehicle | | | | | | |
| (0.3% CMC); | | | | | | |
| p.o.) | | | | | | |
| Group II | 188.7 ± 60.94*** | 120.7 ± 14.48 | 112.5 ± 12.62* | 5.89 ± 0.12 | 106.8 ± 1.83*** | 1.175 ± 0.30*** |
| Paracetamol | | | | | | |
| (200 mg/kg | | | | | | |
| b.w.); p.o. | | | | | | |
| Group III | 65.42 ± 23.50^{###} | 52.23 ± 3.73^{###} | 80.00 ± 5.64 | 6.27 ± 0.14 | 97.65 ± 1.96^{###} | 0.50 ± 0.23^{###} |
| 3-OH-DBP | | | | | | |
| (10 mg/kg | | | | | | |
| b.w.); + | | | | | | |
| Paracetamol; | | | | | | |
| p.o. | | | | | | |
| Group IV | 95.97 ± 29.52 | 85.75 ± 11.59^{###} | 107.1 ± 19.11^{#} | 5.53 ± 0.23* | 57.06 ± 0.90^{###} | 0.48 ± 0.14^{##} |
| 3,8-(OH)₂-DBP | | | | | | |
| (10 mg/kg | | | | | | |
| b.w.); + | | | | | | |
| Paracetamol; | | | | | | |
| p.o. | | | | | | |

Data are expressed as Mean ± SD, n = 6.
p value was obtained by one-way ANOVA followed by post hoc comparison by Newman-Keuls comparison test.
*p < 0.05,
**p < 0.01,
***p < 0.001, in comparison to vehicle control mice.
^{#}p < 0.05,
^{##}p < 0.01,
^{###}p < 0.001, in comparison to paracetamol treated mice.

TABLE 3
Biochemical Parameters After 7 Days of Treatment (Female Mice)

| | Parameters | | | | | |
|---|---|---|---|---|---|---|
| | | | | Total | | Total |
| | ALT | AST | ALP | Protein | Triglyceride | Bilirubin |
| Group | (IU/L) | (IU/L) | (IU/L) | (mg/dl) | (mg/dl) | (mg/dl) |
| Group I | 47.74 ± 9.57 | 51.40 ± 6.4 | 74.58 ± 11.40 | 6.40 ± 0.56 | 78.41 ± 0.55 | 0.187 ± 0.09 |
| Vehicle | | | | | | |
| (0.3% CMC, | | | | | | |
| p.o.) | | | | | | |
| Group II | 155.4 ± 37.73*** | 108.5 ± 7.44*** | 114.6 ± 17.90*** | 6.31 ± 0.23 | 101.00 ± 7.24*** | 1.008 ± 0.18* |
| Paracetamol | | | | | | |
| (200 mg/kg), | | | | | | |
| b.w.; p.o. | | | | | | |
| Group III | 62.32 ± 15.97^{###} | 62.28 ± 12.46^{###} | 82.04 ± 8.96^{#} | 6.60 ± 0.44 | 61.05 ± 1.73^{###} | 0.46 ± 0.26^{#} |
| 3-OH-DBP | | | | | | |
| (10 mg/kg), + | | | | | | |
| Paracetamol; | | | | | | |
| b.w.; p.o. | | | | | | |
| Group IV | 76.91 ± 16.87^{###} | 51.76 ± 3.72^{###} | 105.1 ± 12.40 | 5.41 ± 0.49^{#} | 45.13 ± 0.80^{###} | 0.58 ± 0.30^{#} |
| 3,8-(OH)₂-DBP | | | | | | |
| (10 mg/kg), | | | | | | |
| b.w.; + | | | | | | |
| Paracetamol; | | | | | | |
| p.o. | | | | | | |

Data are expressed as Mean ± SD, n = 6.
p value was obtained by one-way ANOVA followed by post hoc comparison by Newman-Keuls comparison test.
*p < 0.05,
**p < 0.01,
***p < 0.001, in comparison to vehicle control mice.
^{#}p < 0.05,
^{##}p < 0.01,
^{###}p < 0.001, in comparison to paracetamol treated mice.

### Conclusion

In conclusion, this example demonstrates that 3-OH-DBP administration (10 mg/kg body weight orally once daily for 7 days) showed better hepatoprotective activity against paracetamol (200 mg/kg body weight orally once daily for 7 days)-induced liver damage in mice in comparison with 3,8-(OH)₂-DBP administration, in male mice. In female mice, both test compounds (3-OH-DBP and 3,8-(OH)₂-DBP) showed almost the same hepatoprotective activity against paracetamol (administration of 200 mg/kg body weight orally for 7 days).

### Example II

The below example shows hepatoprotective activity of 3-OH-DBP and 3,8-(OH)₂-DBP and treatment and/or prevention of carbon tetrachloride (CCl₄)-induced hepatotoxicity in mice.

### Materials and Methods

3-OH-DBP and 3,8-(OH)₂-DBP were as described in Example I. CCl₄ was in the form of a white colorless liquid.

### Experimental Animals

Swiss albino mice of both sexes, weighing approximately 25-30 g, were obtained from National Research Institute of Ayurveda for Drug Development (Govt. of India), Kolkata, and were housed in polypropylene cages at 22±3° C., relative air humidity of 45 to 55%, with 12.00 hr light and dark cycle (lighting on from 6:00 AM to 6:00 PM) and were provided standard pellet chow (carbohydrate 65.5%, protein 17.6%, fat 6.6%) and distilled water ad libitum. The mice were acclimatized for one week in the laboratory conditions before being used in the experiment. All experiments were conducted between 10.00 hr and 14.00 hr. Principles of laboratory animals care (NIH publication no. 85-23, revised 1985) were followed.

### Carbon Tetrachloride-Induced Hepatotoxicity

Hepatotoxicity was induced by administration of CCl₄ in liquid paraffin (1:2) at the dose of 1.0 ml/kg intraperitoneally once in every 72 hours for 10 days.

### Drug Protocol

The animals were randomly assigned into four groups of six animals each and were administered as shown in Table 4:

TABLE 4
Description of groups

| Group | Administered |
|---|---|
| Group I | Vehicle control (0.3% CMC p.o. (by mouth)) |
| Group II | Carbon tetrachloride CCl₄ (30%) in liquid |
| | paraffin (1:2) (1 ml/kg b.w.) i.p (intraperitoneal) |
| Group III | 3-OH-DBP (10 mg/kg, p.o.) + Carbon tetrachloride |
| | CCl₄ (30%) in liquid paraffin (1:2) (1 ml/kg b.w.) i.p. |
| Group IV | 3,8-(OH)₂-DBP (10 mg/kg b.w, p.o.) + Carbon tetrachloride |
| | CCl₄ (30%) in liquid paraffin (1:2) (1 ml/kg) i.p. |

Test compounds were suspended in 0.3% CMC solutions of distilled water and were administered orally at a dose of 10 mg/kg for 7 days by using an intubation canula and volume of dose was 0.1 ml/10 g body weight. Vehicle control group animals received equivalent volume of the vehicle (0.3% CMC) only.

### Procedure

24 hours after the last sample of blood was collected from retro-orbital plexus under ether anaesthesia, the blood samples were allowed to clot and the serum was separated by centrifugation at 2500 rpm at 37° C. and used for the assay of biochemical marker enzymes (ALT, AST), bilirubin, MDA, and total protein by using commercially available kits (Span Diagnostic Ltd., Surat, India).

### Results and Discussion

Administration of CCl₄ [in liquid paraffin (1:2) at the dose of 1.0 ml/kg [intraperitoneally once in every 72 h for 10 days], produced liver damage in mice (Group II) as indicated by the increase in the levels of hepatic marker enzymes (ALT, AST) in comparison to vehicle control (Group I) (Table 5). Administration of 3-OH-DBP (Group III) and 3,8-(OH)₂-DBP (Group IV) significantly attenuated and reversed the CCl₄-induced hepatotoxicity as evidenced by the significant decrease in the ALT, AST and Bilirubin levels and significant increase in the total protein in comparison to CCl₄ treated group (Group II).

Bilirubin levels are related to the status and function of hepatic cells. In the present study DBPs have been found to significantly reduce bilirubin in the CCl₄ treated groups (Tables 5 and 6, Groups III and IV as compared with Group II).

Also, without being bound by theory, free radicals produced in vivo from CCl₄ attack cell membranes and lead to membrane damage, alteration in the structure and function of the cellular membrane. Thus, increased levels of lipid peroxides, measured herein by increased levels of MDA, are indications of liver damage due to high oxidative stress in CCl₄ intoxicated mice. 3-OH-DBP and 3,8-(OH)₂-DBP significantly lowered CCl₄-induced lipid peroxidation and the results approach or are comparable with those of the vehicle treated group (see Table 5 Groups III and IV, Table 6 Group IV). Without being bound by theory, this signifies the potent antioxidant nature of DBPs.

TABLE 5
Biochemical Parameters After 7 Days of Treatment (Male Mice)

| | Parameters | | | | |
|---|---|---|---|---|---|
| | | | Total | | Total |
| | ALT | AST | Protein | MDA | Bilirubin |
| Groups | (IU/L) | (IU/L) | (mg/dl) | (nmol/ml) | (mg/dl) |
| Group I | 45.15 ± 2.66 | 11.19 ± 2.20 | 3.63 ± 1.57 | 5.58 ± 0.23 | 0.23 ± 0.10 |
| Vehicle | | | | | |
| (0.3% CMC, | | | | | |
| p.o.) | | | | | |
| Group II | 113.53 ± 17.44 | 35.09 ± 3.73 | 2.60 ± 0.41 | 9.39 ± 0.95*** | 1.75 ± 0.30*** |
| CCl₄ (30%), | | | | | |
| b.w.; i.p. | | | | | |
| Group III | 58.21 ± 6.79^{###} | 12.33 ± 3.29^{###} | 3.55 ± 0.31^{#} | 7.04 ± 0.09*^{###} | 0.50 ± 0.23^{###} |
| 3-OH-DBP | | | | | |
| (10 mg/kg), | | | | | |
| b.w., p.o.; + | | | | | |
| CCl₄ (30%, i.p.) | | | | | |
| Group IV | 64.78 ± 4.65*^{###} | 13.04 ± 3.74^{###} | 4.12 ± 0.86^{##} | 6.59 ± 0.82*^{###} | 0.48 ± 0.14^{##} |
| 3,8-(OH)₂-DBP | | | | | |
| (10 mg/kg), | | | | | |
| b.w., p.o.; + | | | | | |
| CCl₄ (30%, i.p.) | | | | | |

Data are expressed as Mean ± SD, n = 6.
p value was obtained by one-way ANOVA followed by post hoc comparison by Newman-Keuls comparison test.
*p < 0.05,
**p < 0.01,
***p < 0.001, in comparison to vehicle control mice.
^{#}p < 0.05,
^{##}p < 0.01,
^{###}p < 0.001, in comparison to carbon tetrachloride treated mice.

TABLE 6
Biochemical Parameters After 7 Days of Treatment (Female Mice)

| | Parameters | | | | |
|---|---|---|---|---|---|
| | | | Total | | Total |
| | ALT | AST | Protein | MDA | Bilirubin |
| Groups | (IU/L) | (IU/L) | (mg/dl) | (nmol/ml) | (mg/dl) |
| Group I | 32.49 ± 1.41 | 11.01 ± 3.18 | 3.65 ± 0.32 | 5.56 ± 0.197 | 0.19 ± 0.04 |
| Vehicle | | | | | |
| (0.3% CMC, | | | | | |
| p.o.) | | | | | |
| Group II | 130.5 ± 22.20 | 35.91 ± 9.39 | 3.79 ± 0.17 | 7.96 ± 0.133 | 1.43 ± 0.27 |
| CCl₄ (30%, | | | | | |
| i.p.), b.w. | | | | | |
| Group III | 67.34 ± 12.07*^{###} | 17.48 ± 5.67^{###} | 3.92 ± 0.88 | 8.13 ± 0.167*** | 0.53 ± 0.21*^{###} |
| 3-OH-DBP | | | | | |
| (10 mg/kg), | | | | | |
| b.w., p.o.; + | | | | | |
| CCl₄ (30%, i.p.) | | | | | |
| Group IV | 60.99 ± 13.49*^{###} | 12.95 ± 2.46^{###} | 4.02 ± 0.30 | 6.53 ± 0.66**^{###} | 0.60 ± 0.18*^{###} |
| 3,8-(OH)₂-DBP | | | | | |
| (10 mg/kg), | | | | | |
| b.w., p.o.; + | | | | | |
| CCl₄ (30%, ip) | | | | | |

Data are expressed as Mean ± SD, n = 6.
p value was obtained by one-way ANOVA followed by post hoc comparison by Newman-Keuls comparison test.
*p < 0.05,
**p < 0.01,
***p < 0.001, in comparison to vehicle control mice.
#p < 0.05,
##p < 0.01,
^{###}p < 0.001, in comparison to carbon tetrachloride treated mice.

### Conclusion

In conclusion, the results of the present study demonstrate that treatment with DBPs showed significant hepatoprotective activity against carbon tetrachloride-induced liver damage in mice. In males and females both 3-OH-DBP and 3,8-(OH)₂-DBP showed significant hepatoprotective activity against carbon tetrachloride-induced liver damage in mice. In females, 3,8-(OH)₂-DBP showed better hepatoprotective activity in comparison to 3-OH-DBP against carbon tetrachloride-induced liver damage.

### Example III

The below example shows hepatoprotective activity of 3-OH-DBP and 3,8-(OH)₂-DBP and treatment and/or prevention of alcohol-induced (ethanol-induced) hepatotoxicity in mice.

### Materials and Methods

3-OH-DBP and 3,8-(OH)₂-DBP were as described in Example I. Alcohol (ethanol) in a stock 400 mg/ml solution was used.

### Experimental Animals

Swiss Albino male and female mice weighing approximately 30±5 g, 2 month old mice were obtained from National Research Institute of Ayurveda for Drug Development (Govt. of India), Kolkata, and were housed in polypropylene cages at 22±3° C., relative air humidity of 45 to 55%, with 12.00 hr light & dark cycle (lighting on from 6:00 AM to 6:00 PM). Mice were provided a standard pellet chow diet (carbohydrate 65.5%, protein 17.6%, fat 6.6%) and distilled water ad libitum. The mice were acclimatized for one week in the laboratory conditions, before being used in the experiment. All experiments were conducted between 10.00 hr and 14.00 hr. Principles of laboratory animals care (NIH publication no. 85-23, revised 1985) were followed.

### Experimental and Treatment Regime

In the present study simultaneous administration of alcohol (ethanol) and treatment with 3-OH-DBP and 3,8-(OH)₂-DBP was done.

### Induction of Alcohol-Induced Hepatotoxicity

Hepatotoxicity was induced by oral administration of alcohol (ethanol: 5 g/kg) from stock 400 mg/ml twice daily for 15 days.

### Drug Regime

Vehicle control animals (“Con Veh”) were administered 0.3% CMC orally for 15 days.

Animals receiving ethanol (“Alc+0.3 CMC”, “3-OH-DBP+Alc”, “3,8-(OH)₂-DBP+Alc”) were administered ethanol in 0.3% CMC orally as discussed above.

3-OH-DBP and 3,8-(OH)₂-DBP were each separately suspended in 0.3% CMC and administered at a dose of 10 mg/kg body weight per orally for 15 days. During the 15 days treatment, body weight, food intake, and water intake were monitored at regular intervals. Following 15 days of treatment physical parameters (relative organ weight) and biochemical parameters (plasma total protein content, ALT, AST, ALP, and ACP) were studied.

### Statistical Analysis

Results were expressed in terms of mean±SD (n=8). The data were subjected to one way ANOVA followed by Tukey's test using GraphPad Prism 4.0 software to establish statistical significance (*p<0.05, **p<0.01, ***p<0.001 vs. vehicle control; # p<0.05, ## p<0.01, ### p<0.001 vs. animals administered ethanol only).

### Results

Tables 7-12 show no significant change was observed in body weight, food intake, or water intake in mice treated with alcohol, 3-OH-DBP+alcohol, or 3,8-(OH)₂-DBP+alcohol, compared with vehicle control mice. Table 13 shows significant effects by 3-OH-DBP and 3,8-(OH)₂-DBP on hepatic biochemical and physical parameters.

### A. Effect on Body Weight

TABLE 7
Effect of 3-OH-DBP and 3,8-(OH)₂-DBP on body weight
(gm) of male mice in alcohol-induced hepatotoxicity

| | | | | 3,8- |
|---|---|---|---|---|
| No. Of | Con Veh | Alc + 0.3 | 3-OH-DBP + | (OH)₂-DBP + |
| Days | (gm) | CMC (gm) | Alc (gm) | Alc (gm) |
| Day 0 | 23.83 ± 0.98 | 30.33 ± 3.33 | 24.33 ± 2.25 | 29.17 ± 2.79 |
| Day 5 | 25.67 ± 1.63 | 28 ± 2.61 | 24 ± 1.55 | 25 ± 3.29 |
| Day 10 | 26.83 ± 2.04 | 27.5 ± 2.26 | 25.5 ± 1.05 | 25.67 ± 2.66 |
| Day 15 | 28.33 ± 2.16* | 27.67 ± 2.50 | 25.3 ± 0.52 | 24.8 ± 2.93 |

In Table 7, body weight (gm) of male 3-OH-DBP+ALC and 3,8-(OH)₂-DBP+ALC treated mice was compared with vehicle control mice and alcohol treated mice. Body weight was significantly (p<0.05) increased in Con Veh group on Day 15 when compared to Day 0. No significant change in body weight was observed (p>0.05) in alcohol (Alc), 3-OH-DBP-treated, or 3,8-(OH)₂-DBP-treated groups.

TABLE 8
Effect of 3-OH-DBP and 3,8-(OH)₂-DBP on body weight
(gm) of female mice in alcohol-induced hepatotoxicity

| | | | | 3,8- |
|---|---|---|---|---|
| No. Of | Con Veh | Alc + 0.3 | 3-OH-DBP + | (OH)₂-DBP + |
| Days | (gm) | CMC (gm) | Alc (gm) | Alc (gm) |
| Day 0 | 21.17 ± 1.17 | 25.5 ± 2.73 | 26.83 ± 1.60 | 25.17 ± 2.32 |
| Day 5 | 22.67 ± 1.21 | 24.83 ± 3.06 | 25.5 ± 1.22 | 23.5 ± 3.02 |
| Day 10 | 23.83 ± 1.33 | 23 ± 3.29 | 24.33 ± 1.97 | 23.67 ± 3.07 |
| Day 15 | 25.00 ± 3.09 | 21 ± 2.64 | 25.17 ± 0.75 | 22.83 ± 2.78 |

In Table 8, body weight (gm) of female 3-OH-DBP+ALC and 3,8-(OH)₂-DBP+ALC treated mice was compared with vehicle control mice and alcohol treated mice. No significant change in body weight was observed (p>0.05).

### B. Effect on Food Intake

TABLE 9
Effect of 3-OH-DBP and 3,8-(OH)₂-DBP on food intake
of male mice in alcohol-induced hepatotoxicity

| | | | | 3,8- |
|---|---|---|---|---|
| No. Of | Con Veh | Alc + 0.3 | 3-OH-DBP + | (OH)₂-DBP + |
| Days | (gm) | CMC (gm) | Alc (gm) | Alc (gm) |
| Day 0 | 20.15 ± 1.33 | 20.32 ± 1.55 | 14.02 ± 0.06 | 17.01 ± 0.75 |
| Day 5 | 20.48 ± 1.52 | 20.83 ± 1.53 | 14.18 ± 0.72 | 17.18 ± 0.85 |
| Day 10 | 19.65 ± 1.05 | 15.98 ± 0.598 | 14.51 ± 0.77 | 17.21 ± 1.11 |
| Day 15 | 20.32 ± 1.02 | 15.76 ± 0.66 | 14.45 ± 0.78 | 17.23 ± 1.16 |

In Table 9, food intake (gm) of male 3-OH-DBP+ALC and 3,8-(OH)₂-DBP+ALC treated mice was compared with vehicle control mice and alcohol treated mice. No significant change in food intake was observed (p>0.05).

TABLE 10
Effect of 3-OH-DBP and 3,8-(OH)₂-DBP on food intake
(gm) of female mice in alcohol-induced hepatotoxicity

| | | | | 3,8- |
|---|---|---|---|---|
| No. Of | Con Veh | Alc + 0.3 | 3-OH-DBP + | (OH)₂-DBP + |
| Days | (gm) | CMC (gm) | Alc (gm) | Alc (gm) |
| Day 0 | 12.55 ± 0.72 | 12.51 ± 0.74 | 8.14 ± 1.67 | 8.52 ± 0.30 |
| Day 5 | 13.00 ± 0.66 | 12.68 ± 0.72 | 8.31.67 | 8.78 ± 1.02 |
| Day 10 | 20 ± 1.13 | 9.32 ± 0.45 | 16.38 ± 1.18 | 16.44 ± 0.47 |
| Day 15 | 19.33 ± 0.86 | 9.65 ± 0.64 | 15.88 ± 0.76 | 16.12 ± 0.66 |

In Table 10, food intake (gm) of female 3-OH-DBP+ALC and 3,8-(OH)₂-DBP+ALC treated mice was compared with vehicle control mice and alcohol treated mice. No significant change in food intake was observed (p>0.05).

### C. Effect on Water Intake

TABLE 11
Effect of 3-OH-DBP and 3,8(OH)₂-DBP on water intake
(ml) of male mice in alcohol-induced hepatotoxicity

| | | | | 3,8- |
|---|---|---|---|---|
| No. Of | Con Veh | Alc + 0.3 | 3-OH-DBP + | (OH)₂-DBP + |
| Days | (ml) | CMC (ml) | Alc (ml) | Alc (ml) |
| Day 0 | 2.09 ± 0.45 | 3.09 ± 0.23 | 2.05 ± 0.23 | 4.92 ± 0.45 |
| Day 5 | 2.17 ± 0.53 | 3.22 ± 0.41 | 2 ± 0.27 | 5.07 ± 0.61 |
| Day 10 | 3.91 ± 0.24 | 2.18 ± 0.40 | 1.93 ± 0.23 | 2.32 ± 0.31 |
| Day 15 | 3.91 ± 0.24 | 2.08 ± 0.28 | 1.98 ± 0.25 | 2.27 ± 0.33 |

In Table 11, water intake (ml) of male 3-OH-DBP+ALC and 3,8-(OH)₂-DBP+ALC treated mice was compared with vehicle control mice and alcohol treated mice. No significant change in water intake (ml) was observed (p>0.05).

TABLE 12
Effect of 3-OH-DBP and 3,8-(OH)₂-DBP on water intake
(ml) of female mice in alcohol-induced hepatotoxicity

| | | | | 3,8- |
|---|---|---|---|---|
| No. Of | Con Veh | Alc + 0.3 | 3-OH-DBP + | (OH)₂-DBP + |
| Days | (ml) | CMC (ml) | Alc (ml) | Alc (ml) |
| Day 0 | 3.05 ± 0.19 | 3.1 ± 0.10 | 4.16 ± 0.60 | 4 ± 0.28 |
| Day 5 | 3.13 ± 0.26 | 3.13 ± 0.12 | 4.32 ± 0.28 | 4.01 ± 0.26 |
| Day 10 | 2.42 ± 0.17 | 2.12 ± 0.20 | 1.85 ± 0.30 | 2.32 ± 0.46 |
| Day 15 | 2.29 ± 0.27 | 2.12 ± 0.29 | 1.84 ± 0.18 | 2.15 ± 0.44 |

In Table 12, water intake (ml) of female 3-OH-DBP+ALC and 3,8-(OH)₂-DBP+ALC treated mice was compared with vehicle control mice and alcohol treated mice. No significant change in water intake (ml) was observed (p>0.05).

### D. Effect on Biochemical and Physical Parameters

Table 13 shows significant effects by 3-OH-DBP and 3,8-(OH)₂-DBP on hepatic biochemical and physical parameters.

TABLE 13
Effect on biochemical and physical parameters of male mice in alcohol-induced hepatotoxicity

| Biochemical Parameters | Con Veh | Alc | 3-OH-DBP + Alc | 3,8-(OH)₂-DBP + Alc |
|---|---|---|---|---|
| Total protein (g/dl) | 2.41 ± 0.384 | 2.64 ± 0.344 | 2.75 ± 0.167 | 3.229 ± 0.187 |
| ALT(SGPT) (IU/L) | 9.37 ± 4.576 | 25.46 ± 9.248* | 11.93 ± 4.364^{#} | 9.98 ± 3.44^{#} |
| AST(SGOT) (IU/L) | 6.21 ± 1.463 | 9.55 ± 1.29* | 5.83 ± 1.75^{#} | 4.51 ± 1.80^{#} |
| ALP(IU/L) | 29.56 ± 6.62 | 32.27 ± 6.99 | 20.20 ± 6.18^{#} | 27.39 ± 6.15 |
| ACP (IU/L) | 3.71 ± 0.71 | 10.92 ± 2.27*** | 5.36 ± 2.34^{#} | 4.29 ± 1.22^{##} |
| Physical Parameters | Con Veh | Alc ± Veh | 3-OH-DBP ± Alc | 3,8-(OH)₂-DBP ± Alc |
| Relative organ weight | | | | |
| (w.r.t body weight) | | | | |
| Liver | 0.0452 ± 0.009 | 0.048 ± 0.003 | 0.044 ± 0.001 | 0.044 ± 0.005 |
| Kidney | 0.015 ± 0.002 | 0.014 ± 0.002 | 0.014 ± 0.001 | 0.016 ± 0.002 |

*p < 0.05,
**p < 0.01,
***p < 0.001 vs. vehicle control;
^{#}p < 0.05,
^{##}p < 0.01,
### p < 0.001 vs. animals administered ethanol only

In Table 13, Total protein, AST, ALT, ALP, ACP of male 3-OH-DBP+ALC and 3,8-(OH)₂-DBP+ALC treated mice was compared with vehicle control and alcohol treated mice.

Plasma ALT was significantly (p<0.05) increased in ALC group when compared with vehicle control group. Treatment with 3-OH-DBP and 3,8-(OH)₂-DBP, significantly (p<0.05) attenuated the alcohol-induced increased ALT indicating hepatoprotective activity

Plasma AST was significantly (p<0.05) increased in ALC group when compared with vehicle control group. Treatment with 3-OH-DBP and 3,8-(OH)₂-DBP, significantly (p<0.05) attenuated the alcohol-induced increased AST indicating hepatoprotective activity

Plasma ACP was significantly (p<0.001) increased in ALC group when compared with vehicle control group. Treatment with 3-OH-DBP and 3,8-(OH)₂-DBP significantly (p<0.05) attenuated the alcohol-induced increased ACP indicating hepatoprotective activity.

No significant (p>0.05) change was observed in plasma total protein content and ALP level, relative liver and kidney organ weight.

TABLE 14
Effect of 3-OH-DBP and 3,8-(OH)₂-DBP on biochemical and physical
parameters of female mice in alcohol-induced hepatotoxicity

| | Con Veh | Alc + Veh | 3-OH-DBP + Alc | 3,8-(OH)₂-DBP + Alc |
|---|---|---|---|---|
| Biochemical Parameters | | | | |
| Total protein(g/dl) | 2.41 ± 0.384 | 2.64 ± 0.344 | 2.75 ± 0.167 | 3.229 ± 0.187 |
| ALT(SGPT) (IU/L) | 9.55 ± 3.05 | 17.55 ± 4.40* | 6.72 ± 2.88^{##} | 6.72 ± 0.727^{##} |
| AST(SGOT) (IU/L) | 5.93 ± 1.45 | 10.31 ± 1.87* | 6.19 ± 1.94^{#} | 3.62 ± 1.27^{##} |
| ALP (IU/L) | 43.80 ± 10.66 | 51.528 ± 8.76 | 32.95 ± 13.06^{#} | 43.12 ± 5.84 |
| ACP (IU/L) | 2.95 ± 0.99 | 9.77 ± 2.85** | 5.58 ± 2.19^{#} | 4.31 ± 2.84^{##} |
| Physical Parameters | | | | |
| Relative organ weight | | | | |
| (w.r.t body weight) | | | | |
| Liver | 0.048 ± 0.004 | 0.050 ± 0.004 | 0.052 ± 0.011 | 0.048 ± 0.001 |
| Kidney | 0.011 ± 0.001 | 0.012 ± 0.001 | 0.013 ± 0.001 | 0.012 ± 0.001 |

*p < 0.05,
**p < 0.01,
***p < 0.001 vs. vehicle control;
^{#}p < 0.05,
^{##}p < 0.01,
### p < 0.001 vs. animals administered ethanol only

As shown in Table 14, total protein, AST, ALT, ALP, ACP of male 3-OH-DBP+ALC and 3,8-(OH)₂-DBP+ALC treated mice were compared with vehicle control and alcohol treated mice.

Plasma ALT was significantly (p<0.05) increased in ALC group when compared with vehicle control group. Treatment with 3-OH-DBP and 3,8-(OH)₂-DBP significantly (p<0.001) attenuated the alcohol-induced increased ALT indicating hepatoprotective activity

Plasma AST was significantly (p<0.05) increased in ALC group when compared with vehicle control group. Treatment with 3-OH-DBP and 3,8-(OH)₂-DBP significantly (p<0.05; p<0.01) attenuated the alcohol-induced increased AST indicating hepatoprotective activity

Plasma ALP did not show significant (p>0.05) increase in ALC group when compared with vehicle control group. Treatment with 3-OH-DBP significantly (p<0.05) decreased plasma ALP level when compared with ALC group. Treatment with 3,8-(OH)₂-DBP did not show significant (p>0.05) change.

Plasma ACP was significantly (p<0.01) increased in ALC group when compared with vehicle control group. Treatment with 3-OH-DBP and 3,8-(OH)₂-DBP, significantly (p<0.05) attenuated the alcohol-induced increased ACP indicating hepatoprotective activity

No significant (p>0.05) change was observed in plasma total protein content, relative liver and kidney organ weight.

### Conclusion

From the above findings it can be concluded that 3-OH-DBP and 3,8-(OH)₂-DBP provide significant hepatoprotective activity against alcohol-induced hepatotoxicity.

### Example IV

### Synergistic Activity of DBPs in Hepatoprotection

Example IV shows hepatoprotective activity of:

3-OH-DBP (2 mg/kg body weight, 10 mg/kg body weight);

3,8-(OH)₂-DBP (10 mg/kg body weight, 50 mg/kg body weight); and

1:5 ratios of 3-OH-DBP and 3,8-(OH)₂-DBP combined
(2 mg 3-OH-DBP/kg body weight+10 mg 3,8-(OH)₂-DBP/kg body weight=12 mg DBPs in a combined 1:5 ratio/kg body weight; and also 10 mg 3-OH-DBP/kg body weight+50 mg 3,8-(OH)₂-DBP/kg body weight=60 mg DBPs in a combined 1:5 ratio/kg body weight)
   and treatment and/or prevention of paracetamol-induced hepatotoxicity (Tables 15-16), alcohol (ethanol)-induced hepatotoxicity (Tables 17-18), and CCl₄-induced hepatotoxicity (Tables 19-20) in mice.

### Materials and Methods

3-OH-DBP and 3,8-(OH)₂-DBP were as described in Example I above. Experimental conditions were as described in Example I (paracetamol study), Example II (CCl₄ study), and Example III (alcohol (ethanol) study), n=4.

### Results

References to synergistic activity and other references to synergy below are not intended as limiting.

### A. Synergistic Hepatoprotective Activity of DBPs Against Paracetamol-Induced Hepatotoxicity

Tables 15 and 16 show the synergistic hepatoprotective activity of DBPs against paracetamol-induced hepatotoxicity

Relative liver weight was significantly (p<0.01) increased in 3-OH-DBP and 3,8-(OH)₂-DBP treated male and female mice. No significant (p>0.05) change in relative kidney weight was observed in the male and female mice.

TABLE 15
Synergistic hepatoprotective activity of DBPs against paracetamol-induced hepatotoxicity in male mice

| | Biochemical Parameters | | | | |
|---|---|---|---|---|---|
| | Total Protein | ALT | AST | ACP | ALP |
| Treatment | (g/dL) | (IU/L) | (IU/L) | (U/L) | (IU/L) |
| Vehicle | 6.43 ± 0.04 | 34.14 ± 5.34 | 33.37 ± 4.64 | 5.75 ± 0.38 | 51.19 ± 4.54 |
| Control | | | | | |
| Paracetamol | 7.36 ± 0.18 | 67.63 ± 3.44** (a) | 102.05 ± 3.72(a) | 18.19 ± 0.46*** (a) | 128.5 ± 2.73***(a) |
| (200 mg/kg, | | | | | |
| b.w.) | | | | | |
| Paracetamol + | 7.28 ± 0.14* | 60.34 ± 4.78** (b) | 71.38 ± 4.63^{#}(b) | 9.69 ± 0.32**,^{##}(b) | 90.17 ± 4.16***,^{#}(b) |
| 3-OH-DBP (2 | | A = (a-b = 7.29) | A = (a-b = 30.67) | A = (a-b = 8.5) | A = (a-b = 38.33) |
| mg/kg, b.w.) | | | | | |
| Paracetamol + | 6.97 ± 0.12 | 49.28 ± 7.23^{#} (c) | 68.23 ± 4.96^{###}(c) | 12.06 ± 0.43***,^{#} (c) | 77.63 ± 6.81^{##} (c) |
| 3,8-(OH)₂-DBP | | B = (a-c = 18.35) | B = (a-c = 33.82) | B = (a-c = 6.13) | B = (a-c = 50.87) |
| (10 mg/kg, | | | | | |
| b.w.) | | | | | |
| Paracetamol + | 7.26 ± 0.07* | 39.34 ± 6.21^{##}(d) | 33.37 ± 7.49^{##}(d) | 5.06 ± 0.71^{###}(d) | 52.55 ± 8.92^{###}(d) |
| 3-OH-DBP + | | C = (a-d = 28.29) | C = (a-d = 68.63) | C = (a-d = 13) | C = (a-d = 75.95) |
| 3,8-(OH)₂-DBP | | C > A + B = | C > A + B = | | |
| (ratio 1:5) (12 | | Synergistic activity | Synergistic activity | | |
| mg/kg, b.w.) | | | | | |
| Paracetamol + | 7.34 ± 0.15* | 51.49 ± 4.49* (e) | 70.35 ± 6.21(e) | 12.75 ± 1.43***,^{#} (e) | 84.75 ± 5.32*,^{#}(e) |
| 3-OH-DBP (10 | | D = (a-e = 16.14) | D = (a-e = 31.7) | D = (a-e = 5.44) | D = (a-e = 43.75) |
| mg/kg, b.w.) | | | | | |
| Paracetamol + | 6.98 ± 0.12 | 47.07 ± 6.69^{#}(f) | 55.69 ± 5.37^{###}(f) | 7.63 ± 0.45^{##}(f) | 79.67 ± 10.18^{###}(f) |
| 3,8-(OH)₂-DBP | | E = (a-f = 20.56) | E = (a-f = 46.36) | E = (a-f = 10.56) | E = (a-f = 48.85) |
| (50 mg/kg, | | | | | |
| b.w.) | | | | | |
| Paracetamol + | 7.27 ± 0.03* | 33.29 ± 3.47^{##} (g) | 32.32 ± 5.55^{###}(g) | 4.13 ± 0.46^{###} (g) | 35.60 ± 5.02^{###} (g) |
| 3-OH-DBP + | | F = (a-g = 34) | F = (a-g = 69.73) | F = (a-g = 14.06) | F = (a-g = 92.92) |
| 3,8-(OH)₂-DBP | | | | | F > D + E = |
| (ratio 1:5) (60 | | | | | Synergistic activity |
| mg/kg, b.w.) | | | | | |

Values are expressed as Mean ± SEM (n = 4). One- way ANOVA followed by Tukey's test was performed to determine significance level.
*p < 0.05,
**p < 0.01,
***p < 0.001 in comparison to vehicle control;
^{#}p < 0.05,
^{##}p < 0.01,
^{###}p < 0.001 in comparison to paracetamol treated male mice.
Values under parentheses indicated numerical exact values of the respective parameters. A, B, C, D, E, F indicated the difference in values from paracetamol treated mice.

TABLE 16
Synergistic hepatoprotective activity of DBPs against
paracetamol-induced hepatotoxicity in female mice

| | Biochemical Parameters | | | | |
|---|---|---|---|---|---|
| | Total Protein | ALT | AST | ACP | ALP |
| Treatment | (g/dL) | (IU/L) | (IU/L) | (U/L) | (IU/L) |
| Vehicle | 6.4 ± 0.29 | 25 ± 2.4 | 30 ± 3.2 | 6.8 ± 0.51 | 31 ± 2.9 |
| Control | | | | | |
| Paracetamol | 6.2 ± 0.30 | 86 ± 10***(a) | 143 ± 22***(a) | 16 ± 0.80***(a) | 98 ± 7.6***(a) |
| (200 mg/kg, | | | | | |
| b.w.) | | | | | |
| Paracetamol + | 6.2 ± 0.16 | 71 ± 9**(b) | 98 ± 10**(b) | 12 ± 0.71***(b) | 77 ± 11***(b) |
| 3-OH-DBP (2 | | A = (a-b = 15) | A = (a-b = 45) | A = (a-b = 4) | A = (a-b = 21) |
| mg/kg, b.w.) | | | | | |
| Paracetamol + | 6.7 ± 0.02 | 47 ± 12^{##} (c) | 79 ± 11*,^{#}(c) | 12 ± 0.63^{##}(c) | 45 ± 7.9^{##}(c) |
| 3,8-(OH)₂-DBP | | B = (a-c = 39) | B = (a-c = 64) | B = (a-c = 4) | D = (a-c = 53) |
| (10 mg/kg, | | | | | |
| b.w.) | | | | | |
| Paracetamol + | 6.5 ± 0.27 | 40 ± 6.2^{##} (d) | 45 ± 4.7^{###}(d) | 9.1 ± 0.83(d) | 51 ± 5.1^{#}(d) |
| 3-OH-DBP + | | C = (a-d = 46) | C = (a-d = 98) | C = (a-d = 6.9) | C = (a-d = 47) |
| 3,8-(OH)₂-DBP | | | | | |
| (ratio 1:5) (12 | | | | | |
| mg/kg, b.w.) | | | | | |
| Paracetamol + | 6.2 ± 0.02 | 50 ± 9.8*,^{#}(e) | 72 ± 6*,^{#}(e) | 8.4 ± 1.2^{#}(e) | 72 ± 6.9**(e) |
| 3-OH-DBP | | D = (a-e = 36) | D = (a-e = 71) | D = (a-e = 7.6) | D = (a-e = 26) |
| (10 mg/kg, | | | | | |
| b.w.) | | | | | |
| Paracetamol + | 5.8 ± 0.30 | 43 ± 2.5^{##}(f) | 66 ± 11*,^{#}(f) | 11 ± 0.41^{#}(f) | 50 ± 11^{#}(f) |
| 3,8-(OH)₂-DBP | | E = (a-f = 43) | E = (a-f = 77) | E = (a-f = 5) | E = (a-f = 48) |
| (50 mg/kg, | | | | | |
| b.w.) | | | | | |
| Paracetamol + | 5.8 ± 0.08 | 30 ± 3.2^{###} (g) | 36 ± 7^{###} (g) | 6.9 ± 0.58^{##} (g) | 39 ± 4.4^{##}(g) |
| 3-OH-DBP + | | F = (a-g = 56) | F(a-g = 107) | F = (a-g = 9.1) | F = (a-g = 59) |
| 3,8-(OH)₂-DBP | | | | | |
| (ratio 1:5) (60 | | | | | |
| mg/kg, b.w.) | | | | | |

Values are expressed as Mean ± SEM (n = 4). One- way ANOVA followed by Tukey's test was performed to determine significance level.
*p < 0.05,
**p < 0.01,
***p < 0.001 in comparison to vehicle control;
^{#}p < 0.05,
^{##}p < 0.01,
^{###}p < 0.001 in comparison to paracetamol treated mice.
Values under parentheses indicated numerical exact values of the respective parameters. A, B, C, D, E, F indicated the difference in values from paracetamol treated mice.

### Conclusion

As shown in Tables 15 and 16, 3-OH-DBP and 3,8-(OH)₂-DBP showed hepatoprotective activity in a dose dependent manner. 3-OH-DBP and 3,8-(OH)₂-DBP (ratio 1:5) shows synergistic hepatoprotective activity in some parameters.

### B. Evaluation of Hepatoprotective Activity of 3-OH-DBP, 3,8-(OH)₂-DBP and its Combination on Alcohol-Induced Hepatotoxicity

Tables 17 and 18 show the synergistic hepatoprotective activity of DBPs against alcohol-induced hepatotoxicity

Relative liver weight was significantly increased in Ursocol treated male mice. Relative liver weight was significantly decreased in 3,8-(OH)₂-DBP treated female mice. No significant change in relative kidney weight was observed in the male or the female mice.

TABLE 17
Synergistic hepatoprotective activity of DBPs against alcohol-induced hepatotoxicity in male mice

| | Biochemical Parameters | | | | |
|---|---|---|---|---|---|
| | Total Protein | ALT | AST | ACP | ALP |
| Treatment | (g/dL) | (IU/L) | (IU/L) | (U/L) | (IU/L) |
| Vehicle | 6.97 ± 0.41 | 28.7 ± 1.67 | 26.1 ± 1.33 | 3.56 ± 0.47 | 61 ± 3.59 |
| Control | | | | | |
| Alcohol (5 | 8.18 ± 0.43 | 106 ± 8.49*** (a) | 60.7 ± 3.72** (a) | 18.6 ± 1.92*** (a) | 134 ± 6.95*** (a) |
| g/kg, b.w.) | | | | | |
| Alcohol (5 | 6.68 ± 0.43 | 74.9 ± 1.66 (b) | 46.5 ± 3.19* (b) | 13.3 ± 1.03***,^{#} (b) | 90.2 ± 6.68*,^{##} (b) |
| g/kg, b.w.) + | | A = (a-b = 31.1) | A = (a-b = 13.5) | A = (a-b = 5.3) | A = (a-b = 43.8) |
| 3-OH-DBP (2 | | | | | |
| mg/kg, b.w.) | | | | | |
| Alcohol (5 | 7.65 ± 0.34 | 53.9 ± 3.99 (c) | 25.6 ± 3.10^{##} (c) | 11.2 ± 1.44***,^{#} (c) | 88.1 ± 15.8 (c) |
| g/kg, b.w.) + | | B = (a-c = 52.1) | B = (a-c = 35.1) | B = (a-c = 7.4) | B = (a-c = 45.9) |
| 3,8-(OH)₂-DBP | | | | | |
| (10 mg/kg, | | | | | |
| b.w.) | | | | | |
| Alcohol (5 | 9.93 ± 0.29** | 49.1 ± 2.09 (d) | 28.0 ± 3.09^{#} (d) | 6.94 ± 0.83*,^{###} (d) | 55.4 ± 2.41^{#} (d) |
| g/kg, b.w.) + | | C = (a-d = 56.9) | C = (a-d = 32.7) | C = (a-d = 25.54) | C = (a-d = 78.6) |
| 3-OH-DBP + | | | | C > A + B = | |
| 3,8-(OH)₂-DBP | | | | Synergistic activity | |
| (ratio 1:5) (12 | | | | | |
| mg/kg, b.w.) | | | | | |
| Alcohol (5 | 8.24 ± 0.43 | 65.4 ± 2.59(e) | 39.9 ± 5.30 (e) | 11.6 ± 0.89***,^{#} (e) | 83.4 ± 4.87^{#} (e) |
| g/kg, b.w.) + | | D = (a-e = 40.6) | D = (a-e = 20.8) | D = (a-e = 7) | D = (a-e = 50.6) |
| 3-OH-DBP (10 | | | | | |
| mg/kg, b.w.) | | | | | |
| Alcohol (5 | 8.60 ± 0.35 | 64.3 ± 2(f) | 24.8 ± 2.19^{##} (f) | 9.19 ± 0.71*,^{##} (f) | 78 ± 7.21^{#} (f) |
| g/kg, b.w.) + | | E = (a-f = 41.7) | E = (a-f = 35.9) | E = (a-f = 9.41) | E = (a-f = 56) |
| 3,8-(OH)₂-DBP | | | | | |
| (50 mg/kg, | | | | | |
| b.w.) | | | | | |
| Alcohol (5 | 8.45 ± 0.08 | 32.2 ± 3.24 (g) | 19.4 ± 2.80^{##} (g) | 3.19 ± 0.47^{###} (g) | 33.9 ± 1.75^{###} (g) |
| g/kg, b.w.) + | | F = (a-g = 73.7) | F = (a-g = 41.3) | F = (a-g = 15.41) | F = (a-g = 100.1) |
| 3-OH-DBP + | | | | | |
| 3,8-(OH)₂-DBP | | | | | |
| (ratio 1:5) (60, | | | | | |
| mg/kg b.w.) | | | | | |
| Alcohol (5 | 8.83 ± 0.16* | 26.6 ± 1.94 | 24.3 ± 3.84^{##} | 3.19 ± 0.36^{###} | 86.1 ± 3.73^{##} |
| g/kg, b.w.) + | | | | | |
| Ursocol (250 | | | | | |
| mg/kg, b.w.) | | | | | |

Values are expressed as mean ± SEM (n = 4). One- way ANOVA followed by Tukey's test was performed to determine significance level.
*p < 0.05,
**p < 0.01,
***p < 0.001 in comparison to vehicle control;
^{#}p < 0.05,
^{##}p < 0.01,
^{###}p < 0.001 in comparison to alcohol treated male mice.
Values under parentheses indicated numerical exact values of the respective parameters. A, B, C, D, E, F indicated the difference in values from alcohol treated mice.

TABLE 18
Synergistic hepatoprotective activity of DBPs against alcohol-induced hepatotoxicity in female mice

| | Biochemical Parameters | | | | |
|---|---|---|---|---|---|
| | Total Protein | ALT | AST | ACP | ALP |
| Treatment | (g/dL) | (IU/L) | (IU/L) | (U/L) | (IU/L) |
| Vehicle | 5.64 ± 0.41 | 37.13 ± 4.75 | 39.78 ± 6.02 | 4.3 ± 0.19 | 57 ± 2.8 |
| Control | | | | | |
| Alcohol (5 | 7.53 ± 0.63* | 88.84 ± 5.56*** (a) | 68.95 ± 4.73* (a) | 21 ± 2.7*** (a) | 140 ± 6.8*** (a) |
| g/kg b.w.) | | | | | |
| Alcohol + | 7.21 ± 0.36 | 85.31 ± 7.89*** (b) | 54.81 ± 8.13* (b) | 19.3 ± 1*** (b) | 106.6 ± 8*** (b) |
| 3-OH-DBP (2 | | A = (a-b = 3.53) | A = (a-b = 14.14) | A = (a-b = 1.97) | A = (a-b = 33.4) |
| mg/kg b.w.) | | | | | |
| Alcohol + | 6.53 ± 0.10 | 45.80 ± 5.76^{#} (c) | 64.09 ± 7.34* (c) | 15.6 ± 0.99***,^{#} | 84 ± 6.5*,^{#} (c) |
| 3,8-(OH)₂-DBP | | B = (a-c = 43.04) | B = (a-c = 4.9) | (c) B = (a-c = 5.67) | B = (a-c = 55.35) |
| (10 mg/kg | | | | | |
| b.w.) | | | | | |
| Alcohol + | 5.73 ± 0.10 | 29.27 ± 3.39^{###} (d) | 29.17 ± 4.18^{###} (d) | 5.3 ± 0.61^{###} (d) | 71 ± 3*,^{#}(d) |
| 3-OH-DBP + | | C = (a-d = 59.57) | C = (a-d = 39.78) | C = (a-d = 15.97) | C = (a-d = 68.7) |
| 3,8-(OH)₂-DBP | | C > A + B = | C > A + B = | | |
| (12 mg/kg | | Synergistic activity | Synergistic activity | | |
| b.w.) | | | | | |
| Alcohol + | 6.53 ± 0.32 | 57.15 ± 3.46*,^{#} (e) | 41.11 ± 6.98^{#} (e) | 12.1±***,^{#} (e) | 79 ± 11*,^{#} (e) |
| 3-OH-DBP (10 | | D = (a-e = 31.69) | D = (a-e = 27.84) | D = (a-e = 8.9) | D = (a-e = 61) |
| mg/kg b.w.) | | | | | |
| Alcohol + | 7.77 ± 0.48* | 31.72 ± 4.07^{##} (f) | 37.13 ± 8.69^{##} (f) | 10.4 ± 0.38**,^{#} (f) | 89 ± 7.9*,^{#} (f) |
| 3-OH-DBP + | | E = (a-f = 57.12) | E = (a-f = 31.82) | E = (a-f = 10.6) | E = (a-f = 45) |
| 3,8-(OH)₂-DBP | | | | | |
| (50 mg/kg | | | | | |
| b.w.) | | | | | |
| Alcohol + | 5.79 ± 0.34 | 24.97 ± 2.23^{###} (g) | 28.29 ± 3.15^{###} (g) | 3.2 ± 0.47^{###} (g) | 56 ± 3.1^{##} (g) |
| 3-OH-DBP ++ | | F = (a-g = 63.87) | F = (a-g = 40.66) | F = (a-g = 17.8) | F = (a-f = 124.4) |
| 3,8-(OH)₂-DBP | | | | | F > D + E = |
| (60 mg/kg | | | | | Synergistic activity |
| b.w.) | | | | | |
| Alcohol (5 | 5.97 ± 0.53 | 26.63 ± 2.85^{###} | 25.39 ± 4.08^{###} | 2.4 ± 0.47^{###} | 49 ± 6.5^{###} |
| g/kg b.w.) + | | | | | |
| Ursocol (250 | | | | | |
| mg/kg b.w.) | | | | | |

Values are expressed as mean ± SEM (n = 4). One-way ANOVA followed by Tukey's test was performed to determine significance level.
*p < 0.05,
**p < 0.01,
***p < 0.001 in comparison to vehicle control;
^{#}p < 0.05,
^{##}p < 0.01,
^{###}p < 0.001 in comparison to alcohol treated mice.
Values under parentheses indicated numerical exact values of the respective parameters. A, B, C, D, E, F indicated the difference in values from alcohol treated mice.

### Conclusion

3-OH-DBP and 3,8-(OH)₂-DBP showed hepatoprotective activity in a dose dependent manner. 3-OH-DBP and 3,8-(OH)₂-DBP (ratio 1:5) showed synergistic hepatoprotective activity in some parameters. The administration of Ursocol® with ethanol (eg bottom row of Tables 17 and 18) restored ALT and AST to approximately those levels shown for vehicle controls in the first row of Tables 17 and 18, as did for instance the 12 mg/kg and 60 mg/kg 1:5 ratios of 3-OH-DBP and 3,8-(OH)₂-DBP in Table 18 and the 60 mg/kg 1:5 ratio in Table 17.

### C. Synergistic Hepatoprotective Activity of DBPs Against CCL₄-Induced Hepatotoxicity

Tables 19 and 20 show the synergistic effect of DBPs on CCl₄-induced hepatotoxicity.

Relative liver weight was significantly increased in 3,8-(OH)₂-DBP treated male mice. No significant change in relative kidney weight was observed in the male and female mice.

TABLE 19
Synergistic hepatoprotective activity of DBPs against CCL₄-induced hepatotoxicity in male mice

| | Biochemical Parameters | | | | |
|---|---|---|---|---|---|
| | Total Protein | ALT | AST | ACP | ALP |
| Treatment | (g/dL) | (IU/L) | (IU/L) | (U/L) | (IU/L) |
| Vehicle | 5.6 ± 0.25 | 36 ± 3.8 | 42 ± 1.5 | 9.8 ± 1.2 | 51 ± 13 |
| Control | | | | | |
| CCl₄ (1 | 6.1 ± 0.34 | 80 ± 4.7**(a) | 103 ± 4.4*** (a) | 86 ± 9.9***(a) | 142 ± 19***(a) |
| ml/kg, b.w.) | | | | | |
| CCl₄ + | 5.6 ± 0.37 | 72 ± 3.4**(b) | 70 ± 4##(b) | 49 ± 3.6***,#( b) | 79 ± 12#(b) |
| 3-OH-DBP (2 | | A = (a-b = 8) | A = (a-b = 33) | A = (a-b = 37) | A = (a-b = 63) |
| mg/kg, b.w.) | | | | | |
| CCl₄ + | 6.3 ± 0.36 | 68 ± 5.1** (c) | 70 ± 4.4##(c) | 48 ± 7.2***,#(c) | 89 ± 5.1*,,#(c) |
| 3,8-(OH)₂-DBP | | B = (a-c = 12) | B = (a-c = 33) | B = (a-c = 38) | B = (a-c = 53) |
| (10 mg/kg, | | | | | |
| b.w.) | | | | | |
| CCl₄ + | 8.2 ± 2.9 | 36 ± 4.4**,## (d) | 24 ± 3.7###(d) | 40 ± 4.8***,# (d) | 63 ± 3.3##(d) |
| 3-OH-DBP + | | C = (a-d = 44) | C = (a-d) = 79 | C = (a-d = 46) | C = (a-d = 79) |
| 3,8-(OH)₂-DBP | | C > A + B = | C > A + B = | | |
| (ratio 1:5) (12 | | Synergistic activity | Synergistic activity | | |
| mg/kg, b.w.) | | | | | |
| CCl₄ + | 6.3 ± 0.46 | 67 ± 3.6** (e) | 69 ± 2.7## (e) | 47 ± 2.6***,# (e) | 78 ± 6.9#(e) |
| 3-OH-DBP (10 | | D = (a-e = 13) | D = (a-e = 34) | D = (a-e = 39) | D = (a-e = 64) |
| mg/kg, b.w.) | | | | | |
| CCl₄ + | 6 ± 0.40 | 60 ± 2.5*,# (f) | 58 ± 1.9###(f) | 46 ± 2.5***,# (f) | 68 ± 8##(f) |
| 3,8-(OH)₂-DBP | | E = (a-f = 20) | E = (a-f = 45) | E = (a-f = 20) | E = (a-f = 74) |
| (50 mg/kg, | | | | | |
| b.w.) | | | | | |
| CCl₄ + | 6 ± 0.73 | 20 ± 3.7### (g) | 13 ± 1.7###(g) | 21 ± 3**,## (g) | 44 ± 7.5###(g) |
| 3-OH-DBP + | | F = (a-g = 20) | F = (a-g = 98) | F = (a-g = 65) | F = (a-g = 98) |
| 3,8-(OH)₂-DBP | | | F > D + E = | F > D + E = | |
| (ratio 1:5) (60 | | | Synergistic activity | Synergistic activity | |
| mg/kg, b.w.) | | | | | |
| CCl₄ + | 6 ± 0.55 | 21 ± 2.6### | 18 ± 3.4### | 22 ± 4.4**,## | 60 ± 9.9## |
| Ursocol (250 | | | | | |
| mg/kg, b.w.) | | | | | |

Values are expressed as mean ± SEM (n = 4). One- way ANOVA followed by Tukey's test was performed to determine significance level.
*p < 0.05,
**p < 0.01,
***p < 0.001 in comparison to vehicle control;
#p < 0.05,
##p < 0.01,
###p < 0.001 in comparison to paracetamol treated male mice.
Values under parentheses indicated numerical exact values of the respective parameters. A, B, C, D, E, F indicated the difference in values from CCl4 treated mice.

TABLE 20
Synergistic hepatoprotective activity of DBPs against CCL₄-induced hepatotoxicity in female mice

| | Biochemical Parameters | | | | |
|---|---|---|---|---|---|
| | Total Protein | ALT | AST | ACP | ALP |
| Treatment | (g/dL) | (IU/L) | (IU/L) | (U/L) | (IU/L) |
| Vehicle | 3.9 ± 0.30 | 42 ± 6.3 | 34 ± 34 | 11 ± 3.1 | 45 ± 10 |
| Control | | | | | |
| CCl₄ (1 | 2.5 ± 0.41 | 87 ± 8.6** (a) | 105 ± 4.2*** (a) | 80 ± 6.8***(a) | 90 ± 6.6*** (a) |
| ml/kg, b.w.) | | | | | |
| CCl₄ + | 4.7 ± 0.59 | 81 ± 7** (b) | 90 ± 4.2*** (b) | 48 ± 5.6***,^{##} (b) | 75 ± 13** (b) |
| 3-OH-DBP (2 | | A = (a-b = 6) | A = (a-b = 15) | A = (a-b = 32) | A = (a-b = 15) |
| mg/kg, b.w.) | | | | | |
| CCl₄ + | 4.9 ± 0.21 | 68 ± 6.5* (c) | 57 ± 8* (c) | 48 ± 4**,^{##} (c) | 89 ± 5.1** (c) |
| 3,8-(OH)₂-DBP | | B = (a-c = 19) | B = (a-c = 48) | B = (a-c = 32) | B = (a-c = 1) |
| (10 mg/kg, | | | | | |
| b.w.) | | | | | |
| CCl₄ + | 9.4 ± 1*** | 61 ± 4.3*,^{#} (d) | 47 ± 7.6^{###} (d) | 24 ± 1.4^{###} (d) | 60 ± 9.9^{#} (d) |
| 3-OH-DBP + | | C = (a-d = 26) | C = (a-d = 58) | C = (a-d = 56) | C = (a-d = 30) |
| 3,8-(OH)₂-DBP | | C > A + B = | | | C > A + B = |
| (ratio 1:5) (12 | | Synergistic activity | | | Synergistic activity |
| mg/kg, b.w.) | | | | | |
| CCl₄ + | 7.2 ± 0.23** | 75 ± 7.6*(e) | 69 ± 4.9*,^{##} (e) | 55 ± 4.1***,^{#} (e) | 78 ± 6.9** (e) |
| 3-OH-DBP (10 | | D = (a-e = 12) | D = (a-e = 36) | D = (a-e = 26) | D = (a-e = 12) |
| mg/kg, b.w.) | | | | | |
| CCl₄ + | 6.6 ± 0.16** | 62 ± 3.6*^{#} (f) | 41 ± 14^{###}(f) | 28 ± 5.1*,^{###} (f) | 68 ± 8^{#} (f) |
| 3,8-(OH)₂-DBP | | E = (a-f = 25) | E = (a-f = 64) | E = (a-f = 52) | E = (a-f = 22) |
| (50 mg/kg, | | | | | |
| b.w.) | | | | | |
| CCl₄ + | 5.9 ± 0.57 | 47 ± 4.8^{##} (g) | 38 ± 4.5^{###}(g) | 19 ± 2.4^{###} (g) | 39 ± 5.1^{###} (g) |
| 3-OH-DBP + | | F = (a-g = 40) | F = (a-g = 67) | E = (a-g = 61) | F = (a-g = 51) |
| 3,8-(OH)₂-DBP | | F > D + E = | | | F > D + E = |
| (ratio 1:5) (60 | | Synergistic activity | | | Synergistic activity |
| mg/kg, b.w.) | | | | | |
| CCl₄ + | 6 ± 0.22 | 50 ± 5.6^{#} | 49 ± 6.8^{###} | 29 ± 3.3**,^{#} | 45 ± 6.9^{##} |
| Ursocol (250 | | | | | |
| mg/kg, b.w.) | | | | | |

Values are expressed as mean ± SEM (n = 4). One- way ANOVA followed by Tukey's test was performed to determine significance level.
*p < 0.05,
**p < 0.01,
***p < 0.001 in comparison to vehicle control;
^{#}p < 0.05,
^{##}p < 0.01,
^{###}p < 0.001 in comparison to paracetamol treated mice.
Values under parentheses indicated numerical exact values of the respective parameters. A, B, C, D, E, F indicated the difference in values from CCl4 treated mice.

### Conclusion

From the above findings it can be concluded that 3-OH-DBP and 3,8-(OH)₂-DBP showed hepatoprotective activity in a dose dependent manner and 3-OH-DBP and 3,8-(OH)₂-DBP (ratio 1:5) showed synergistic hepatoprotective activity in some parameters.

The use of the terms “a,” “an,” “the,” and similar referents in the context of describing the present invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%; in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied. All method steps described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise stated. No language in the specification should be construed as indicating any non-claimed element as essential to the practice of the invention.

While in the foregoing specification the present invention has been described in relation to certain embodiments thereof, and many details have been put forth for the purposes of illustration, it will be apparent to those skilled in the art that the invention is susceptible to additional embodiments and that certain of the details described herein can be varied considerably without departing from the basic principles of the invention.

The present invention may be embodied in other specific forms without departing from the spirit or essential attributes thereof, and, accordingly, reference should be made to the appended claims, rather than to the foregoing specification, as indicating the scope of the invention.

1. A method of treating and/or preventing hepatotoxicity in a subject comprising the steps of:
a) providing a composition comprising 3-OH-dibenzo-alpha-pyrone (3-OH-DBP) and/or 3,8-(OH)₂-dibenzo-alpha-pyrone (3,8-(OH)₂-DBP); and
b) administering the composition to the subject in an effective amount to deliver the 3-OH-DBP and/or 3,8-(OH)₂-DBP to the subject's liver to act on the liver and improve one or more abnormal hepatic parameters and/or maintain one or more hepatic parameters in the subject.

2. The method of claim 1, wherein the effective amount of 3-OH-DBP is a daily dose of about 0.5 mg 3-OH-DBP/kg of the subject's body weight to about 75 mg 3-OH-DBP/kg of the subject's body weight.

3. The method of claim 2, wherein said daily dose is about 1.0 mg/kg to about 50 mg/kg.

4. The method of claim 2, wherein said daily dose is about 1.5 mg/kg to about 25 mg/kg.

5. The method of claim 1, wherein the effective amount of 3,8-(OH)₂-DBP is a daily dose of about 0.5 mg 3,8-(OH)₂-DBP/kg of the subject's body weight to about 75 mg 3,8-(OH)₂-DBP/kg of the subject's body weight.

6. The method of claim 5, wherein said daily dose is about 1.0 mg/kg to about 50 mg/kg.

7. The method of claim 5, wherein said daily dose is about 1.5 mg/kg to about 25 mg/kg.

8. The method of claim 1, wherein the composition comprises a combination of 3-OH-DBP and 3,8-(OH)₂-DBP in a ratio of about 1:3 to about 1:7.

9. The method of claim 8, wherein the ratio is 1:5 and wherein the effective amount is a combined (3-OH-DBP+3,8-(OH)₂-DBP) daily dose of about 0.5 mg (3-OH-DBP+3,8-(OH)₂-DBP)/kg of the subject's body weight to about 75 mg (3-OH-DBP+3,8-(OH)₂-DBP)/kg of the subject's body weight.

10. The method of claim 9, wherein said daily dose is about 1.0 mg/kg to about 50 mg/kg.

11. The method of claim 9, wherein said daily dose is about 1.5 mg/kg to about 25 mg/kg.

12. The method of claim 1, wherein the composition is administered daily for 7 days or more.

13. The method of claim 1, wherein said hepatic parameter is serum ALT, serum AST, serum ALP, serum ACP, serum bilirubin, serum triglycerides, and/or serum MDA.

14. The method of claim 1, wherein the hepatotoxicity is from ethanol, paracetamol, and/or carbon tetrachloride.

15. The method of claim 1, wherein said treating and/or preventing hepatotoxicity is treating and/or preventing liver damage from an alcohol-related disorder and/or drug-induced liver injury.

16. A method of providing hepatoprotection in a subject comprising the steps of:
a) providing a composition comprising 3-OH-DBP and/or 3,8-(OH)₂-DBP; and
b) administering the composition to the subject in an effective amount to deliver the 3-OH-DBP and/or 3,8-(OH)₂-DBP to the tissues and bloodstream of the subject's liver to protect the liver from damage.

17. The method of claim 16, wherein the effective amount is a daily dose of about 0.5 mg 3-OH-DBP/kg of the subject's body weight to about 75 mg 3-OH-DBP/kg of the subject's body weight.

18. The method of claim 17, wherein said daily dose is about 1.0 mg/kg to about 50 mg/kg.

19. The method of claim 17, wherein said daily dose is about 1.5 mg/kg to about 25 mg/kg.

20. The method of claim 16, wherein the effective amount is a daily dose of about 0.5 mg 3,8-(OH)₂-DBP/kg of the subject's body weight to about 75 mg 3,8-(OH)₂-DBP/kg of the subject's body weight.

21. The method of claim 20, wherein said daily dose is about 1.0 mg/kg to about 50 mg/kg.

22. The method of claim 20, wherein said daily dose is about 1.5 mg/kg to about 25 mg/kg.

23. The method of claim 16, wherein the effective amount is a combined (3-OH-DBP+3,8-(OH)₂-DBP) daily dose of about 0.5 mg (3-OH-DBP+3,8-(OH)₂-DBP)/kg of the subject's body weight to about 75 mg (3-OH-DBP+3,8-(OH)₂-DBP)/kg of the subject's body weight.

24. The method of claim 23, wherein said daily dose is about 1.0 mg/kg to about 50 mg/kg.

25. The method of claim 23, wherein said daily dose is about 1.5 mg/kg to about 25 mg/kg.

26. The method of claim 16, wherein the composition comprises a combination of 3-OH-DBP and 3,8-(OH)₂-DBP in a ratio of about 1:3 to about 1:7.

27. The method of claim 26, wherein the ratio is 1:5 and wherein the effective amount is a combined (3-OH-DBP+3,8-(OH)₂-DBP) daily dose of about 0.5 mg (3-OH-DBP+3,8-(OH)₂-DBP)/kg of the subject's body weight to about 75 mg (3-OH-DBP+3,8-(OH)₂-DBP)/kg of the subject's body weight.

28. The method of claim 27, wherein said daily dose is about 1.0 mg/kg to about 50 mg/kg.

29. The method of claim 27, wherein said daily dose is about 1.5 mg/kg to about 25 mg/kg.

30. The method of claim 16, wherein the composition is administered daily for 7 days or more.

31. The method of claim 16, wherein said hepatoprotection attenuates, reverses, or eliminates hepatotoxicity by improving at least one hepatic parameter of the subject from an abnormal hepatotoxic level to a normal healthy level or to a level approaching a normal healthy level for the subject or on average for the subject's species and optionally gender; and/or
wherein said hepatoprotection avoids or minimizes hepatotoxicity from hepatotoxic insult by maintaining at least one hepatic parameter of the subject, after said hepatotoxic insult, at a normal healthy level or a level approaching a normal healthy level for the subject or on average for the subject's species and optionally gender, as compared with a typical change to an abnormal hepatotoxic level from such hepatic insult in the subject's species and optionally gender.

32. The method of claim 16, wherein said hepatic parameter is serum ALT, serum AST, serum ALP, serum ACP, serum bilirubin, serum triglycerides, and/or serum MDA.

33. The method of claim 16, wherein the hepatoprotection protects the subject's liver from injury from an alcohol-related disorder and/or drug-induced liver injury.

34. The method of claim 33, wherein the hepatoprotection is from ethanol, paracetamol, and/or carbon tetrachloride.
